(19) United States
(12) Patent Application Publication
(10) Pub. No.: US 2025/0241775 A1
(43) Pub. Date: Jul. 31, 2025

(54) ORTHOPEDIC DEVICE HAVING A LOCK MECHANISM

(71) Applicant: Ossur Iceland ehf, Reykjavik (IS)

(72) Inventor: David SANDAHL, Reykjavik (IS)

(21) Appl. No.: 19/040,220

(22) Filed: Jan. 29, 2025

Related U.S. Application Data
(60) Provisional application No. 63/627,285, filed on Jan. 31, 2024.

(51) Int. Cl. A61F 5/01; A61F 5/05

(52) U.S. Cl. CPC A61F 5/0106; A61F 5/05; A61F 2005/0158

(57) ABSTRACT

A pivot assembly incorporates a lock mechanism positioned within a compound recess on a first strut. The locking mechanism is arranged to establish a secure connection in either an engaged position or a disengaged position, depending on its alignment with multiple notches located on a second strut and its specific position within the compound recess. A spring element is integrated with the lock mechanism, enabling it to interact with plurality recesses formed within the compound recess according to the engaged and disengaged states of the lock mechanism.

### FIELD OF THE DISCLOSURE

The disclosure relates to orthopedic devices, including immobilization braces for the knee and elbow, to aid user outcomes following surgery or injury. The orthopedic device includes an improved lock mechanism to arrest and hold a rotational position.

### BACKGROUND

In orthopedic devices, and more particularly adjustable knee or elbow braces or supports, it is desirable that the brace can limit the range of motion of a lower limb relative to the upper limb both as to bending in flexion and in extension of the lower limb relative to the upper limb. Various immobilization brace arrangements have been proposed, and these have included, such as for the knee, upper struts for extending along the thigh, and lower struts for extending along the lower leg or calf. These braces or supports are often arranged in a “double upright” configuration because they are provided both on the inside or medial side of the leg and the outer or lateral side. The medial and lateral struts are normally padded and provided with straps to hold them in place by circumferential tension. Pivoting arrangements are provided for coupling the upper and lower struts, and stops are provided to limit the knee's extension and flexion.

Common indications for immobilization of the knee include anterior-, posterior-, medial-, and lateral-collateral ligament repairs and injuries, meniscal repairs, tibial plateau fractures, patellar tendon repairs, osteochondral repairs, condylar fractures, and sprains or strains of the knee. Common indications for immobilization of the elbow include fracture stabilization of the elbow, distal humerus, proximal radius or ulna, muscle, tendon, and ligament repairs, reconstructions or injuries, range of motion control of the elbow post-injury, and elbow hypertension.

Many prior art immobilization braces have shortcomings because they tend to be bulky, have complicated adjustment mechanisms, cause undue limb discomfort, or need to be ergonomically configured. These prior art braces also may not hold proper or desired configuration during use and can often inadvertently adjust to a position or configuration not desired or intended.

Providing a drop lock to arrest or maintain the immobilization brace in a desired angular position is of particular interest. Past efforts are found in U.S. Pat. No. 11,096,816, granted on 24 Aug. 2021; U.S. Pat. No. 10,806,620, granted on 20 Oct. 2020; and U.S. Pat. No. 7,534,220, granted 19 May 2009, wherein each of these patents is incorporated herein by their entirety. These patents teach a pivot assembly or hinge with a drop lock or lock mechanism that can quickly and easily lock down the relative pivoting motions of the upper and lower or first and second struts. A sliding drop lock button is preferably on the pivot assembly so that when slid into its locking position in a recess formed along an outer cover of the pivot assembly, the drop lock interlocks the upper and lower struts to inhibit their pivoting action. Indicia may be provided, allowing the wearer to know at what angle the pivot assembly should be locked. A preferable range is −10 to 40 degrees.

However, despite these past efforts and the considerable strain and stress applied to a drop lock due to the weight of the user and resistance of the pivot assembly, the drop lock can deteriorate over time, particularly a spring or similar device, or insufficiently maintain position, or yet further be too challenging for a user to adjust. Moreover, it may become challenging to mass produce the drop locks in a manner that is both cost-effective and efficacious in function. Therefore, there is a need for an orthopedic brace with an improved locking mechanism that overcomes possible shortcomings known in the prior art.

### SUMMARY

The embodiments of the disclosure relate to orthopedic braces that overcome the drawbacks in the prior art, particularly regarding a lock mechanism.

According to an embodiment, a pivot assembly or hinge for an orthopedic device includes a first strut having a first strut head, the first strut head defining a compound recess, and a second strut having a strut head. The second strut head has a curved or circular shape and defines a plurality of notches about a circumference. A lock mechanism is disposed of within the compound recess and arranged to lock in an engaged position and a disengaged position relative to the plurality of notches according to a position within the compound recess.

In an embodiment, the compound recess defines an entry or channel opening from the first end or peripheral surface of the first strut head, which is arranged to cooperate with a peripheral surface of the second strut head. The compound recess defines a longitudinal axis bisecting the channel. The compound recess further defines a first recess depending on the end of the channel opposite the first end of the first strut head and along the longitudinal axis. The first recess may have a circular, semi-circular, or arcuate periphery on the first side (e.g., anterior side) of the longitudinal axis. The compound recess may have a second recess with a circular, semi-circular, or arcuate periphery on the second side (e.g., posterior side) of the longitudinal axis, depending on the first recess along the longitudinal axis.

The pivot assembly may include a spring element adapted to urge against a periphery of the first recess and a surface of the lock mechanism in the engaged position and is adapted to urge against another surface of the lock mechanism and bias against the second recess in the disengaged position. The spring element may be formed from a flexible and resilient polymeric material and, more preferably, from Delrin. The spring element may have a circular shape that encircles a portion of the lock mechanism to urge against the first and second sides of the lock mechanism, whether the spring element is biased against the first or second recess, respectively.

The combination of the compound recess and the circular spring provides an advantage over the prior art in that the lock mechanism is easily adjustable yet securely remains in its different positions. The pairing of the spring element and the lock mechanism provided with the compound recess allows for secure placement of the lock mechanism while still offering a user-friendly mechanism to toggle between locked and unlocked positions. The circular spring and the nature of its composition are advantageous in that they can be readily reproduced and lead to a sufficiently robust design that can endure repeated use and wear of the orthopedic device. The disclosed pivot assembly offers a simplified, more streamlined solution for a drop lock mechanism that requires fewer parts for manufacturing and assembly.

These and other features, aspects, and advantages of the present disclosure will become better understood regarding the following description, appended claims, and accompanying drawings.

### BRIEF DESCRIPTION OF THE DRAWINGS

FIG. 1 is a perspective view of a prior art orthopedic device or knee immobilizer having a drop lock.

FIG. 2 is an exploded view of an upright assembly in the orthopedic device or knee immobilizer of FIG. 1.

FIG. 3 is an exploded view of a pivot assembly having a lock mechanism according to an embodiment of the disclosure.

FIG. 4 is a plan view of a lock body in the lock mechanism of FIG. 3.

FIG. 5 is an elevational view of the lock body of FIG. 4.

FIG. 6 is a plan view of a plate in the lock mechanism of FIG. 3.

FIG. 7 is a partial plan view of the first end of the first strut in the lock mechanism of FIG. 3.

FIG. 8 is a schematic plan view of the lock mechanism in an engaged position.

FIG. 9 is a schematic plan view of the lock mechanism in a disengaged position.

FIGS. 10A-10D are perspective views of exemplary lock bodies for the lock mechanism.

The drawings are not necessarily drawn to scale but instead are drawn to provide a better understanding of the components and are not intended to be limiting in scope but to provide exemplary illustrations. It will be noted that while not clearly depicted as such, the features central holes, as depicted with exemplary reference character(s) 140, should be aligned along the axis A-A.

### DETAILED DESCRIPTION OF VARIOUS EMBODIMENTS

For further ease of understanding the exemplary embodiments of an orthopedic device in an immobilization or post-operative knee or elbow brace as disclosed, these terms may be used with the features of the exemplary embodiments. The term “upper” refers to a location on top or above a median portion (such as in a pivot assembly proximate to the knee). Likewise, the term “lower” refers to a location below a median portion (such as in a pivot assembly proximate to the knee) and is in contrast to the term “upper.”

The terms “proximal” and “distal” may refer to relative locations of parts or places on the limbs. Proximal may refer to something closer to the torso, while distal refers to parts and places away from the torso. The femur is proximal to the knee, which is proximal to the ankle and the toes.

The terms “inner” or “inside” may have their ordinary meaning and refer to an inside portion or location adjacent to or more proximate to a leg or knee. The terms “outer” or “outside” have their ordinary meaning and refer to a relative location opposite an inner or inside portion and is the side or surface typically on the outside of or proximate to the outside of the device.

The terms “medial” and “lateral” are relative and may indicate location near the midsagittal plane or midline. Therefore, elements near the midline are “medial,” and those further from the midline are considered “lateral.” The term “central” denotes the area along the midline of a joint dividing and sharing regions of the medial and lateral regions. The medial side of the knee is the inside part or side nearest to the other knee, while the lateral side of the knee faces away from the center of the body and is farthest from the other knee.

The terms “rigid,” “flexible,” and “resilient” may distinguish characteristics of portions of certain features of the orthopedic brace. The term “rigid” should denote that an element of the device generally lacks flexibility. Within the context of support members that are “rigid,” it should indicate that they do not lose their overall shape when force is applied, and they may break if bent with sufficient force. The term “flexible” should denote that features are capable of repeated bending such that the features may be bent into retained shapes or do not retain a general shape but continuously deform when force is applied. The term “resilient” qualifies such flexible features as returning to an initial general shape without permanent deformation. The term “semi-rigid” connotes properties with some flexibility or resiliency.

It will be understood that the usage of ordinals, such as “first,” “second,” etc., are nominally used as associative denotations but are not necessarily provided to denote spatial location. Rather, the ordinal merely associates one element with another element or distinguishes one element from another element.

The term “compound recess” means that an opening or recess has at least two recesses or cuts at different locations and/or different shapes, thereby forming part of the whole of a compound recess.

Various embodiments described herein are in the exemplary form of an immobilization or post-operative knee brace. However, it will be understood that similar embodiments and components as described, considered individually or in combination with other components, may be used for an orthopedic device in an elbow brace, ankle brace, shoulder brace, or other brace in which motion control and/or motion limiting of a body joint is desired. The components do not have to be used with the other components described herein.

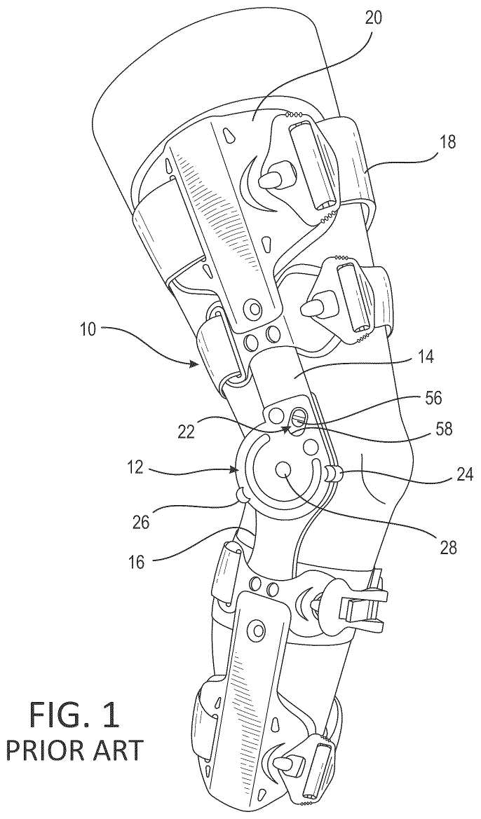

FIG. 1 illustrates an orthopedic device 10 as a knee immobilizer and is likewise shown and described in U.S. Pat. No. 11,096,816. The orthopedic device 10 includes a pivot assembly 12 (e.g., a hinge) connecting first and second struts 14, 16 carrying strap and shell assemblies 18, 20. The pivot assembly 12 includes a drop lock assembly 22 and first and second pivot stops 24, 26 to limit flexion and extension of the knee. The pivot assembly 12 pivots about a central pivot 28 that may be embodied by a pin. It is about the central pivot 28 that the drop lock 22 assembly can limit the rotation of the pivot assembly 12. The drop lock assembly 22 includes a button 56 for moving within a slot 58 between unlocked and locked positions.

The first strut, 14, may be referenced as a femoral or proximal strut (extending along the femur or upper leg bone), and the second strut, 16, may be referenced as a tibial or distal strut (extending along the tibia or principal lower leg bone). Preferably, the struts are preferably formed from a material (e.g., aluminum) that provides sufficient rigidity to support and brace the knee joint, although they may be malleable to allow optional bending by a clinician for better conformation to a user's leg. When worn, however, the struts do not bend or yield to the user's leg, but instead remain stiff or rigid when worn by the user.

The pivot assembly enables rotation of the first strut 14 relative to the lower second strut 16 about a central axis (shown here as surrounding the central pivot 28 or pin). The pivot stops are configurable to limit the relative rotation of the first and second struts 14, 16 to limit extension and flexion of a user's knee when the brace has been donned. For a post-operative user, it is often desirable to limit the bending of the knee in the extension direction (straightening out the leg) and the flexion direction (bending the leg).

The drop lock assembly is evidenced by a drop lock button configured to lock the relative pivoting of the first and second struts quickly and easily. When the drop lock is moved from an unlocked or disengaged position to a locking or engaged position, the drop lock functions to lock the first strut 14, so rotation of the first and second struts is prevented.

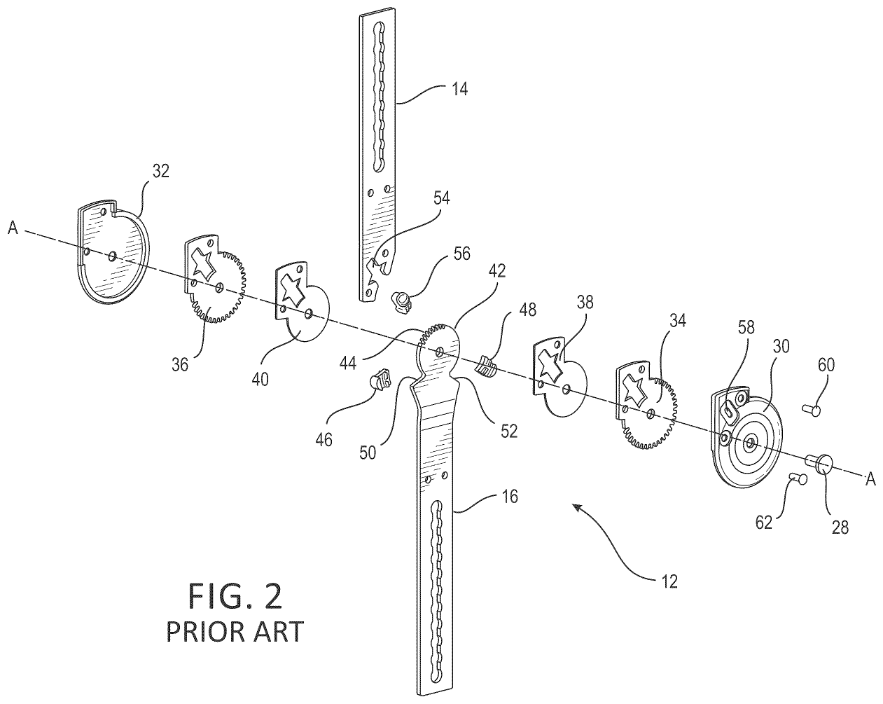

FIG. 2 illustrates an exploded view of the pivot assembly 12, which may include the first and second struts 14, 16, as their relative movement is controlled by the pivot assembly 12. The pivot assembly 12 may also be considered to omit the first and second struts 14, 16. As depicted in FIG. 2, the pivot assembly 12 includes an outer or first cover 30, an outer or first plate 34, an outer or first bearing 38, a strut head 42 from which the second strut 16 extends, an inner or second bearing 40, an inner or second plate 36, and an inner or second cover 32, all of which are positioned about a central axis A-A as connected by a central pivot 28 (e.g., pin or fastener) extending through central holes 140. The first and second covers 30, 32, and first and second plates 34, 36 may define a flat, disc-like shape. The central pivot 28 extends through each component's central holes to attach the pivot assembly 12 components to the second strut 16. Fasteners 60, 62 are used to fasten the components of the pivot assembly 12 to the first strut 14. As shown, the first and second plates 34, 36 and the first and second bearings 38, 40 are rigidly secured to the upper strut 14, while the lower strut 16 is rotatable relative to the first and second plates 34, 36 about the central axis A-A.

While the first and second plates 34, 36 and first and second covers 30, 32 are depicted and described as being discretely separate elements, they may be formed monolithically. For example, the outer plate and outer cover may be formed together as a single element to form a unitary element constructed from the same material or a combination of materials. The combination of the outer plate and outer cover and the inner plate and inner cover, whether considered unitary or discretely separate, may be referred to as a pivoting element. For example, the combination of the outer plate and the outer cover may be referred to as an outer pivoting element, and the inner plate and the inner cover may be referred to as an inner pivoting element.

Pivot stops 46, 48 are rotatable relative to the first and second plates 34, 36. The pivot stops 46, 48 are configured to selectively be engaged with the notches of the first and second plates 34, 36 to delimit flexion and extension of the second strut 16 relative to the first strut 14 as the stop edges 50, 52 abut against the pivot stops 46, 48 to prevent further rotation of the second strut 16 past the pivot stops 46, 48. The drop lock 22 is disposed within a drop lock recess 54 that extends through the first and second plates 34, 36, first and second bearings 38, 40, and the first strut 14. The drop lock 22 is configured to be selectively engaged with the notches 44 of the second strut 16 to lock rotation of the first and second struts 14, 16 relative to one another.

The first and second bearings 38, 40 are preferably formed from a polymer material such as a polyamide (e.g., nylon) or polytetrafluoroethylene. The first and second bearings 38, 40 are positioned between the first and second plates 34, 36 and the strut head 42 of the second strut 16. In this configuration, the first and second bearings 38, 40 can provide smooth rotation of the second strut 16 relative to the first strut 14. This beneficially provides a user with a more comfortable and natural feel during flexion and/or extension motions of the knee by avoiding any catching or binding between the first and second plates 34, 36, and the strut head 42. In addition, the first and second bearings 38, 40 protect the adjacent components to which they are applied, increasing the durability and usability of the knee brace.

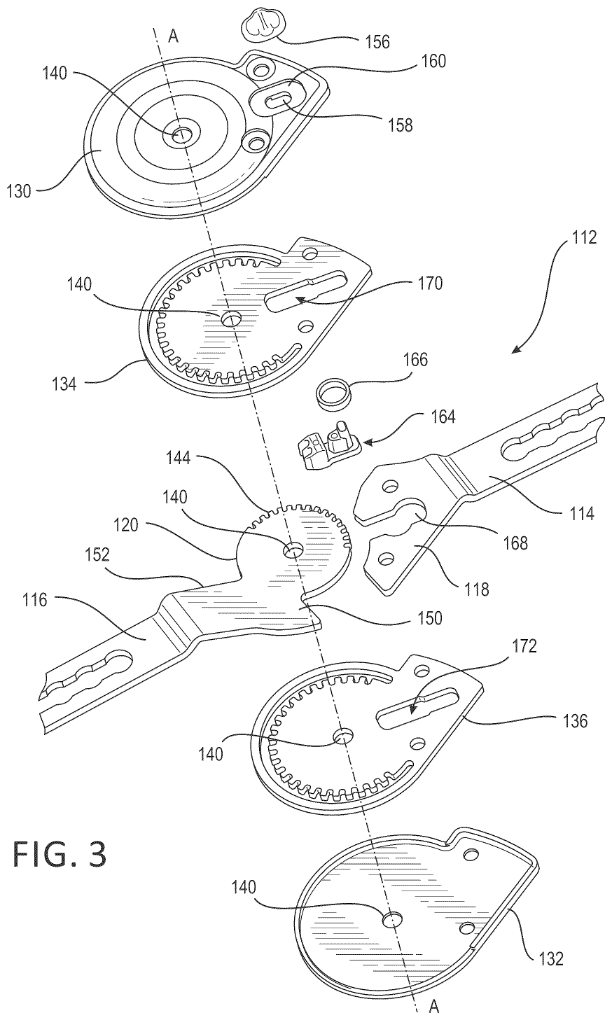

While not depicted in the embodiment of FIG. 3, the aforementioned bearings may be included in the pivot assembly 112 of FIG. 3 in that the bearings are configured and dimensioned to correspond to the depicted plates, yet for each of understanding FIG. 3, the bearings are omitted. The bearings may define a sliding slot corresponding to the sliding slot 170 of plates.

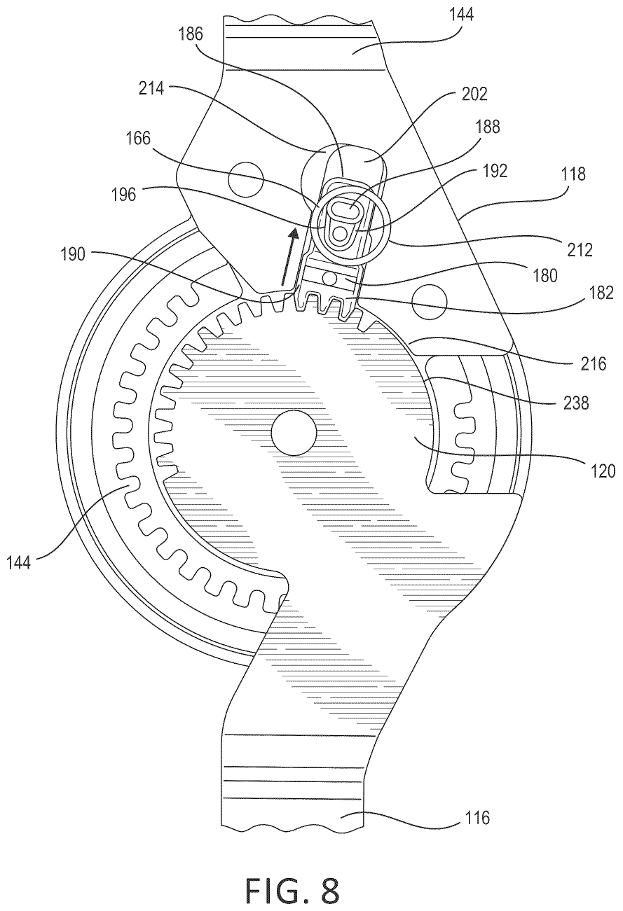

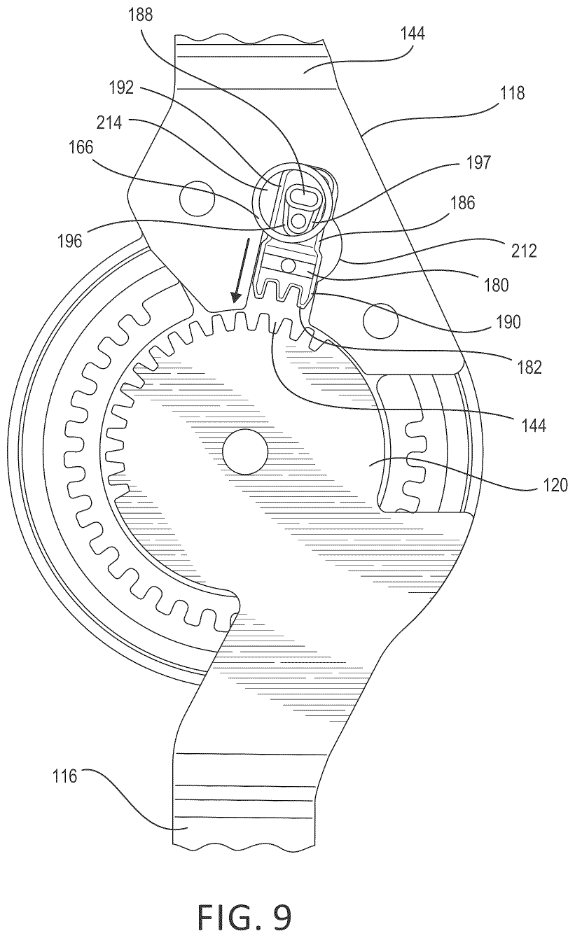

According to the disclosure, FIGS. 3-7 disclose an improved pivot assembly 112 and components thereof, for the orthopedic device 10 of FIG. 1. The pivot assembly 112 has a central axis A-A comprising a first or upper strut 114 having a first strut head 118. The first strut head 118 defines a compound recess 168 for receiving a lock mechanism 164 in multiple positions. The pivot assembly 112 further includes a second strut 116 having a strut head 120. The second strut head 120 has a curved or circular shape and defines a plurality of notches 144 disposed about a circumference thereof. The lock mechanism 164 is disposed of within the compound recess 168 and is arranged to lock in an engaged position and a disengaged position, as shown in FIGS. 8 and 9, respectively, relative to the plurality of notches 144 according to a position within the compound recess 168.

The compound recess 168 defines an entry or channel 210 opening from a first end or first peripheral surface 216 of the first strut head 118. The first peripheral surface 216 is arranged to cooperate with a second peripheral surface 238 of the second strut head 120. In an embodiment, the second peripheral surface 238 is defined by the curve or circular shape of the second strut head 120 and the first peripheral surface 216 defines an opposing curve or circular shape. In an embodiment, the longitudinal axis B-B is coincident to a radius of the curve or circular shape of the second strut head 120.

The compound recess 168 defines a longitudinal axis B-B bisecting the channel 210. The compound recess 168 defines a first recess 212 depending on an end of the channel 210 opposite the first end of the first strut head 118 and along the longitudinal axis B-B. The first recess 212 has a circular, semi-circular, or arcuate periphery on the first side of the longitudinal axis B-B. The compound recess 168 has a second recess 214 with a circular, semi-circular, or arcuate periphery on the second side of the longitudinal axis B-B) and, depending on the first recess 212, along the longitudinal axis (B-B). The circular, semi-circular, or arcuate periphery for each recess 212, 214 corresponds to circular, semi-circular, or arcuate periphery of the spring element 166.

The first strut 114 defines a first stop surface 218 to limit the rotation of the pivot assembly 112 in a first rotational direction. A second stop surface 220 will limit the rotation of the pivot assembly 112 in a second rotational direction. The second strut 116 defines a first stop surface 150 to limit rotation of the pivot assembly 112 in the first rotational direction and is adapted to abut the first stop surface 218 of the first strut 114. A second stop surface 152 limits the rotation of the pivot assembly 112 in the second rotational direction by abutting the second stop surface 220.

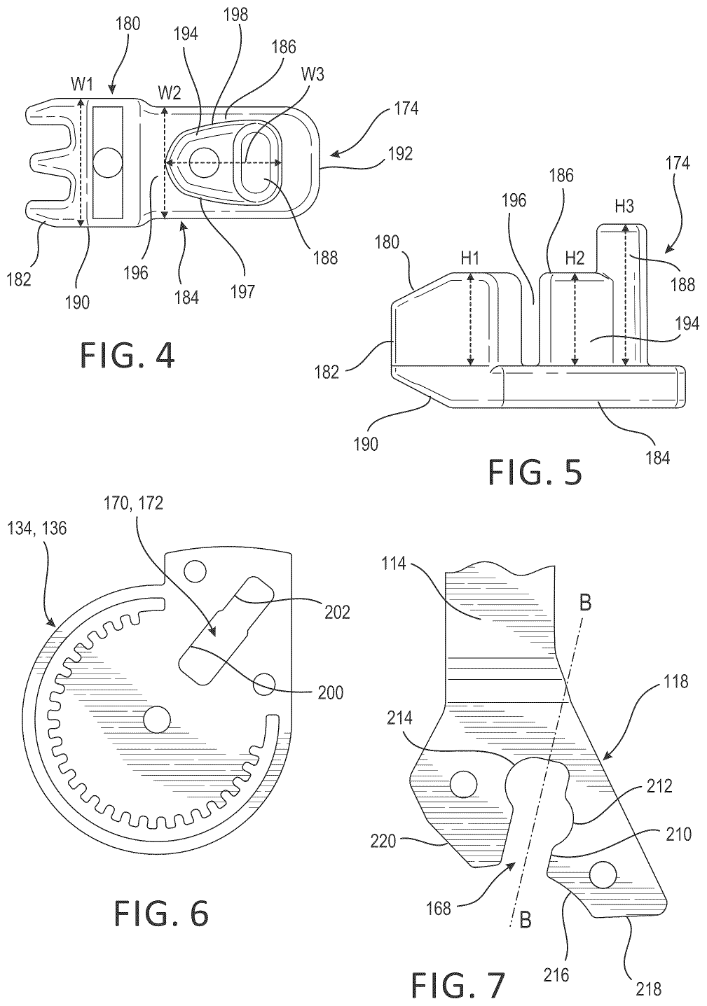

Referring to FIGS. 4 and 5, the lock mechanism 164 includes a lock body 174 arranged to slide within the compound recess 168 to and from the first end or peripheral surface 216 along the longitudinal axis B-B. The lock mechanism 164 has a lock body 174 defining a head portion 180 having a first width W1 and the head portion 180 forming at least one tooth 182 arranged to selectively engage at least one of the notches 144 of the second strut 116.

The channel 210 is configured and dimensioned to receive the head portion 180 such that a periphery 190 of the head portion 180 is configured and dimensioned to generally match the size or width of the channel 210, thereby directing only linear movement of the head portion 180 within the channel 210 that is parallel to the longitudinal axis B-B.

The lock body 174 further defines a base portion 184 extending from the end of the head portion 180, and having a second width W2. The base portion 184 defines a retainer 186 protruding upwardly or relatively perpendicularly to the longitudinal axis B-B. The retainer 186 has a periphery 194 tapering in width W3 toward the head portion 180 defined along the longitudinal axis B-B. The periphery 194 of the retainer 186 tapers uniformly on opposed first and second sides 197, 198 of the retainer 186 and is bisected by a longitudinal axis of the retainer 186 along the width W3. The tapered periphery 194 of the retainer 186 allows for a smoother transition to a locked position by more narrowly focusing the point of contact to displace the spring element 166 along the longitudinal axis B-B and through the compound recess 168.

The periphery 190 of the head portion 180 is greater than the width of the periphery of the base portion 184 as defined perpendicularly to the longitudinal axis B-B. A gap 196 is formed between the head portion 180 and the retainer 186. A shaft 188 protrudes a height H3 from the retainer 186 in the direction of a height H1, H2 of the head portion 180 and/or retainer 186. In an embodiment, the height H3 of the shaft 188 is greater than the height H1, H2 of the head portion 180 and/or retainer 186. The shaft 188 is arranged to carry a button 156 adapted for providing actuation of the lock body 174 within the recess 168.

As shown in FIG. 3, the first cover 130 defines a shaft slot 158 arranged parallel with the longitudinal axis B-B and is configured and dimensioned to accommodate the shaft 188 therein during sliding movement of the lock body 174 within the recess 168. The first cover, 130, covers the first side of the pivot assembly 112. A second cover 132 covers a second side of the pivot assembly 112 and sandwiches the pivot assembly 112 with the first cover 130. The first cover 130 defines a button recess 160 corresponding to and encircling the shaft slot 158. The button recess 160 is configured and dimensioned to accommodate the sliding movement of the button 156 for actuating the lock body 174 in the engaged and disengaged positions.

FIGS. 3, 8, and 9 illustrate a spring element 166 adapted to urge against a periphery of the first recess 212 and the retainer 186 in the engaged position, as in FIG. 8, and is adapted to urge against the retainer 186 and the second recess 214 in the disengaged position, as in FIG. 9. The spring element 166 passes through the gap 196. As observed in FIG. 8, the spring element 166 provides added stability to the pivot assembly 112 in a locked position by interfacing with the lock body 174 at the strut 188 of the base portion 184 and with the head portion 180 through the gap 196. Thus, displacement of the lock body 174 along the longitudinal axis B-B is met with resistance of the spring element 166 at two points of contact to secure the pivot assembly 112 in a locked position.

The spring element 166 is formed from a flexible and resilient polymeric material, preferably from Delrin. “Delrin” is known as polyoxymethylene or acetal, polyacetal, and polyformaldehyde. It is an engineering thermoplastic used in precision parts requiring high stiffness, low friction, and excellent dimensional stability.

The spring element 166 preferably has a circular shape and encircles the retainer 186, extending through the gap 196, to urge against the first and second sides 197, 198 or shaft 188 of the retainer 186 whether the spring element 166 is biased against the first recess 212 or the second recess 214, respectively. In an embodiment, the first recess 212 is formed on an anterior surface of the compound recess 168 and the second recess 214 is formed on a posterior surface of the compound recess 168, the first recess 212 being distal and offset from the second recess 214 along the longitudinal axis B-B.

As in the prior art pivot assembly, the pivot assembly 112 includes first and second plates 134, 136 for pivot stops. Unlike in the prior art, the first and second plates 134, 136 each define a sliding slot 170, 172 having a first slot region 200 configured and dimensioned to accommodate sliding movement of the head portion 180 of the lock body 174, and a second slot region 202 configured and dimensioned to accommodate sliding movement of the base portion 184 of the lock body 174, the first slot region 200 having an approximate width to accommodate a first width W1 of the head portion 180. The second slot region 202 having an approximate width accommodate a second width W2 of the base portion 184. The second slot region 202 is arranged to correspond to the periphery 192 of the base portion 184 and prevents rotational deviation or displacement away from the longitudinal axis B-B.

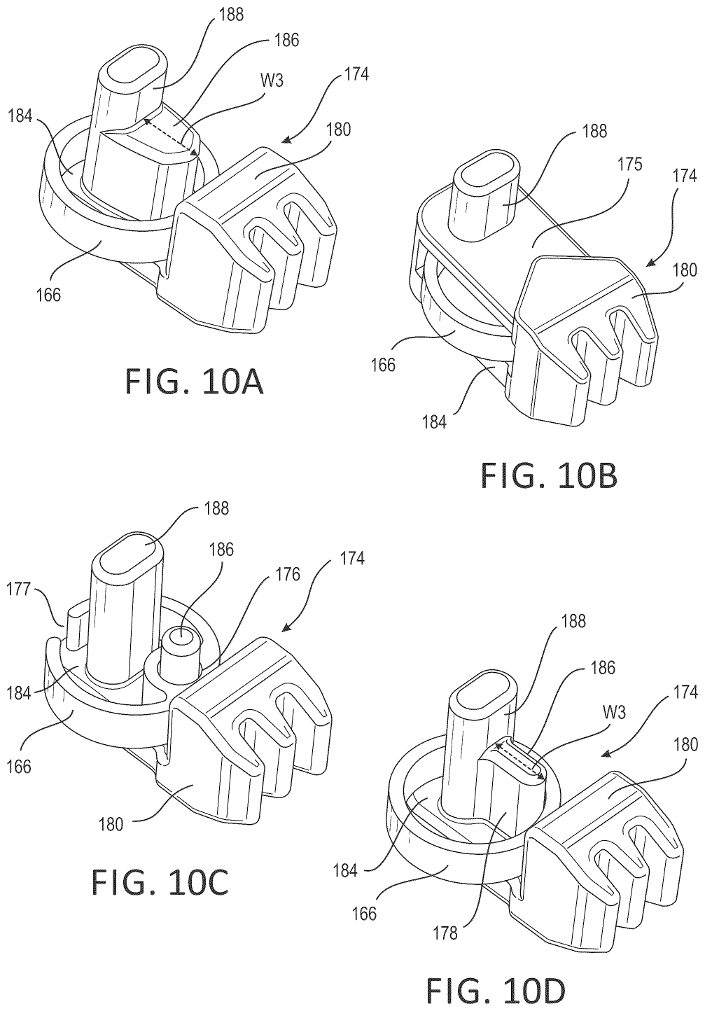

FIG. 10A illustrates a perspective view of the lock body 174 described in FIGS. 4-5. As observed, the retainer 186 has a periphery 194 tapering in width W3 toward the head portion 180 defined along the longitudinal axis B-B. The tapered periphery 194 of the retainer 186 allows for a smoother transition to a locked position by narrowly focusing the point of contact to displace the spring element 166 along the longitudinal axis B-B and through the compound recess 168.

FIGS. 10B-10D depict alternative embodiments of the lock body 174. FIG. 10B illustrates the spring element 166 housed between the base portion 184 and a covering 175. The covering 175 extends from the periphery 190 of the head portion 180 toward the periphery 192 of the base portion 184. The shaft 188 of the retainer 186 extends from the base portion 184 through an opening of the covering 175, e.g., the shaft 188 protrudes a height H3 from the retainer 186. The covering 175 retains the spring element 166 from being displaced in a direction parallel to the shaft 188 and perpendicular to the longitudinal axis B-B.

FIG. 10C illustrate the spring element 166 having an aperture 176 radially formed along in inner arc of the spring element 166. The aperture 176 is arranged to receive the retainer 186. The retainer 186 is formed between the gap 196 and shaft 188 (e.g., another gap is formed between the retainer 186 and shaft 188). By arranging the retainer 186 to extend through the aperture 176 of the spring element 166, the spring element 166 is configured to pivot about the retainer 186 and between first and second recesses 212, 214 of the recess 168. In an embodiment, the spring element 166 may include a split 177 formed along an arc of the spring element 166. The split 177 may be on a side of the spring element 166 opposite the aperture 176 and proximal to the periphery 192 of the base portion 184.

Similar to FIG. 4, FIG. 10D illustrates a lock body 174 having a retainer 186 integrated with the shaft 188. In FIG. 10C, the retainer 186 features a linear section 178 wherein opposed first and second sides (e.g., sides 197, 198) are parallel with the longitudinal axis B-B along the width W3 of the retainer 186.

While the orthopedic device has mostly been described in relation to an immobilization or post-operative knee brace, it will be understood that the principles, features, and/or components described may be extended to other types of orthopedic and prosthetic devices.

It is to be understood that not necessarily all objects or advantages may be achieved under any embodiment of the disclosure. Those skilled in the art will recognize that a lock mechanism may be embodied or carried out to achieve or optimize one advantage or group of advantages as taught herein without achieving other objects or advantages as taught or suggested herein.

The skilled artisan will recognize the interchangeability of various disclosed features. Besides the variations described herein, other known equivalents for each feature can be mixed and matched by one of ordinary skill in this art to build and use a palmar strap assembly under the principles of the present disclosure. The skilled artisan will understand that the features described herein may be adapted to other methods and types of prosthetic devices/applications.

1. A pivot assembly for an orthopedic device having a first longitudinal axis or central axis, comprising:
a first strut having a first strut head, the first strut head defining a compound recess;
a second strut having a strut head, the second strut head having a curved or circularly shape and defines a plurality of notches disposed about a circumference thereof;
a lock mechanism disposed within the compound recess and arranged to lock in an engaged position and a disengaged position relative to the plurality of notches according to a position within the compound recess;
wherein the compound recess defines an entry or channel opening from a first end or peripheral surface of the first strut head and is arranged to cooperate with a peripheral surface of the second strut head;
wherein the compound recess defines a second longitudinal axis bisecting the channel.

2. The pivot assembly of claim 1, wherein the compound recess further defines a first recess depending from an end of the channel opposite the first end of the first strut head and along the longitudinal axis, the first recess located on a first side of the longitudinal axis, and the compound recess having a second recess located on a second side of the longitudinal axis.

3. The pivot assembly of claim 2, wherein the first recess has a circular or semi-circular or arcuate periphery on the first side of the second longitudinal axis, and the second recess has a circular or semi-circular or arcuate periphery on the second side of the second longitudinal axis.

4. The pivot assembly of claim 1, wherein the lock mechanism includes a lock body arranged to slide within the compound recess to and from the first end or peripheral surface along the second longitudinal axis.

5. The pivot assembly of claim 1, wherein the first strut defines a first stop surface to limit rotation of the pivot assembly in a first rotational direction, and a second stop surface to limit rotation of the pivot assembly in a second rotational direction.

6. The pivot assembly of claim 5, wherein the second strut defines a first stop surface to limit rotation of the pivot assembly in the first rotational direction and is adapted to abut the first stop surface of the first strut, and a second stop surface to limit rotation of the pivot assembly in the second rotational direction by abutting the second stop surface.

7. The pivot assembly of claim 1, wherein the lock mechanism has a lock body defining a head portion forming at least one tooth arranged to selectively engage at least one of the notches of the second strut.

8. The pivot assembly of claim 7, wherein the channel is configured and dimensioned to receive the head portion such that a periphery of the head portion is configured and dimensioned to generally match a size or width of the channel, thereby directing only linear movement of the head portion within the channel that is parallel to the second longitudinal axis.

9. The pivot assembly of claim 8, wherein the lock body further defines a base portion extending from an end of the head portion, the base portion defining a retainer protruding upwardly or relatively perpendicularly to the second longitudinal axis.

10. The pivot assembly of claim 9, wherein the periphery of the head portion is greater than a width of a periphery of the base portion as defined perpendicularly to the second longitudinal axis.

11. The pivot assembly of claim 9, wherein the retainer has a periphery tapering in width toward the head portion defined along the second longitudinal axis.

12. The pivot assembly of claim 10, wherein the periphery of the retainer tapers uniformly on opposed first and second sides of the retainer and is bisected by a second longitudinal axis of the retainer along the width.

13. The pivot assembly of claim 9, wherein a gap is formed between the head portion and the retainer.

14. The pivot assembly of claim 9, wherein a shaft protrudes a third height from the retainer in the direction of first or second heights of the head portion and/or retainer, respectively, the shaft is arranged to carry a button adapted for providing actuation of the lock body within the recess.

15. The pivot assembly of claim 14, further comprising a first cover defining a shaft slot arranged parallel with the second longitudinal axis and is configured and dimensioned to accommodate the shaft therein during sliding movement of the lock body within the recess, wherein the first cover covers a first side of the pivot assembly.

16. The pivot assembly of claim 15, wherein the first cover defines a button recess corresponding to and encircling the shaft slot, the button recess is configured and dimensioned to accommodate the sliding movement of the button for actuating the lock body in the engaged and disengaged positions.

17. The pivot assembly of claim 1, wherein the compound recess further defines a first recess depending from an end of the channel opposite the first end of the first strut head and along the second longitudinal axis, the first recess located on a first side of the second longitudinal axis, and the compound recess having a second recess located on a second side of the longitudinal axis;
wherein the lock mechanism has a lock body defining a head portion forming at least one tooth arranged to selectively engage at least one of the notches of the second strut;
wherein the lock mechanism further comprises a spring element adapted to urge against a periphery of the first recess and the retainer in the engaged position and is adapted to urge against the retainer and the second recess in the disengaged position, wherein the spring may likewise pass through the gap.

18. The pivot assembly of claim 17, wherein the spring element is formed from a flexible and resilient polymeric material.

19. The pivot assembly of claim 17, wherein the spring element has a circular shape and encircles the retainer to urge against the first and second sides or shaft of the retainer whether the spring element is biased against the first recess or the second recess, respectively.

20. The pivot assembly of claim 1, wherein the lock mechanism has a lock body defining a head portion forming at least one tooth arranged to selectively engage at least one of the notches of the second strut;
wherein the channel is configured and dimensioned to receive the head portion such that a periphery of the head portion is configured and dimensioned to generally match a size or width of the channel thereby directing only linear movement of the head portion within the channel that is parallel to the second longitudinal axis;
wherein the lock body further defines a base portion extending from an end of the head portion, the base portion defining a retainer protruding upwardly or relatively perpendicularly to the second longitudinal axis;
the pivot assembly further comprising first and second plates for pivot stops, the first and second plates each defining a sliding slot having a first slot region configured and dimensioned to accommodate sliding movement of the head portion of the lock body, and a second slot region configured and dimensioned to accommodate sliding movement of the base portion of the lock body, the first slot region having an approximate width to accommodate a first width of the head portion, and the second slot region having an approximate width accommodate a second width of the base portion.
